(19) United States
(12) Patent Application Publication
(10) Pub. No.: US 2004/0186483 A1
(43) Pub. Date: Sep. 23, 2004

(54) Implant driver apparatus and bone joining device

(76) Inventor: George W. Bagby, Spokane, WA (US)

Correspondence Address: WELLS ST. JOHN P.S., 601 W. FIRST AVENUE, SUITE 1300, SPOKANE, WA, 99201, US

(21) Appl. No.: 10/394,749

(22) Filed: Mar. 22, 2003

(51) Int. Cl. A61B 17/58

(52) U.S. Cl. 606/99; 623/17.11

(57) ABSTRACT

An apparatus is provided for driving an implant. The apparatus includes a drive body having a leading end with an implant engaging surface and a plurality of drive elements contiguous with the implant engaging surface. The drive elements extend radially outwardly from the implant engaging surface, adjacent the leading end. The drive elements are configured to engage with corresponding apertures along a trailing end of an implant.

### TECHNICAL FIELD

The present invention pertains to surgical joining of bone bodies. More particularly, the present invention relates to instruments, implants, and methods for implant insertion, instant fixation and staged bone fusion or arthrodesis of bone bodies, such as spinal vertebrae.

### BACKGROUND OF THE INVENTION

Techniques are known for driving a foreign object between bone bodies to encourage fusion and arthrodesis between the bone bodies. For example, dowels of bone were driven via tapping between adjacent vertebrae according to one early technique for achieving arthrodesis, referred to as the well-known Cloward Technique for use in the human cervical spine. Threads were later added to the dowels of bone according to Otero-Vich German Patent Application Number 3,505,567, published Jun. 5, 1986. Subsequently, U.S. Pat. No. 4,501,269 to Bagby, issued Feb. 26, 1985, taught the use of a metal dowel in the form of a bone basket which is inserted between adjacent vertebrae. Subsequently, threads were added to such metal bone baskets, or cages.

In the cases of smooth bone dowels and bone baskets (or cages), the dowel or basket is axially tapped into place using a driver tool. In the case of threaded bone dowels and threaded bone baskets (or cages), the object is torqued into place using a driver tool. For the case of smooth bone baskets inserted between vertebrae, axial tapping typically imparts distraction between the adjacent vertebrae, putting the intervertebral disk annulus on stretch.

U.S. Pat. No. 6,447,545 B1 to Bagby, issued Sep. 10, 2002, discloses one tool and implant for driving a threaded implant between a pair of adjacent vertebrae having prepared bone beds. Such tool has movable parts that include a pair of extendable and retractable drive pins for engaging within drive holes provided in a rear open edge of the implant. However, such tool is relatively complicated, using threaded parts and apertures for the pins and retraction mechanism, and is prone to being contaminated with soft tissue and bodily fluids. Hence, post-operative cleaning is somewhat complicated and costly. Additionally, threaded portions are susceptible to being clogged with soft tissue. Furthermore, such a construction proves to be somewhat difficult to clean and sterilize.

Accordingly, improvements are needed to eliminate the above-described deficiencies.

### SUMMARY OF THE INVENTION

An instrument and implant are provided for inserting and removing a bone joining implant between a pair of bone bodies, such as inserting and removing a threaded vertebral body implant between a pair of adjacent vertebrae. The implant includes oblique apertures that mate with drive pins on the instrument during insertion. Additionally, the same features facilitate removal of the implant when occasionally necessary.

According to one aspect, an apparatus is provided for driving an implant. The apparatus includes a drive body having a leading end with an implant engaging surface and a plurality of drive elements contiguous with the implant engaging surface. The drive elements extend radially outwardly from the implant engaging surface, adjacent the leading end. The drive elements are configured to engage with corresponding apertures along a trailing end of an implant.

According to another aspect, a spinal fusion device is provided. The spinal fusion device includes a cylindrical fusion body having an outer surface including threads, a trailing portion with an open trailing end, and a plurality of drive apertures formed in an inner surface of the open trailing end. Each drive aperture has an enlarged drive slot spaced from the trailing end and a narrowed entrance slot provided in the trailing end and communicating with the enlarged drive slot.

### BRIEF DESCRIPTION OF THE DRAWINGS

Preferred embodiments of the invention are described below with reference to the following accompanying drawings.

FIG. 1 is a perspective view of an implant system including an implant insertion tool usable for inserting and threading a novel implant within the prepared bone beds of FIGS. 5-9.

FIG. 2 is a simplified, elevational and sagittal view of the implant and the implant insertion tool prior to loading the implant onto the insertion tool and prior to insertion.

FIG. 3 is a simplified, elevational and sagittal view of the implant and the implant insertion tool after loading the implant onto the insertion tool and prior to insertion, illustrating the implant rotated clockwise (from the perspective of a user of the tool) into an insertion drive position atop the insertion tool.

FIG. 4 is a simplified, elevational and sagittal view of the implant and the implant insertion tool illustrating the implant rotated counter-clockwise (from the perspective of a user of the tool) into a removal drive position atop the insertion tool.

FIG. 5 is a simplified, sagittal and partial centerline view of the implant and the implant insertion tool of FIG. 1.

FIG. 6 is a simplified, sagittal and centerline view of the implant and the implant insertion tool of FIG. 1 after insertion.

FIG. 7 is a surgical time simplified sagittal view of the implant of FIG. 1 received within the prepared bone beds of adjacent vertebrae and containing bone fragments immediately following implantation.

FIG. 8 is a healed time simplified sagittal view of the implant of FIG. 1 received within the prepared bone beds of adjacent vertebrae and illustrating the vertebrae following bone remodeling and reorganization and showing arthrodesis.

FIG. 9 is a coronal view of the implant and healed bone comprising vertebrae and taken along line 9-9 of FIG. 8, and showing arthrodesis.

FIG. 10 is a perspective view of an alternative construction implant insertion tool usable for inserting and threading (via torquing) a novel implant within prepared bone beds.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

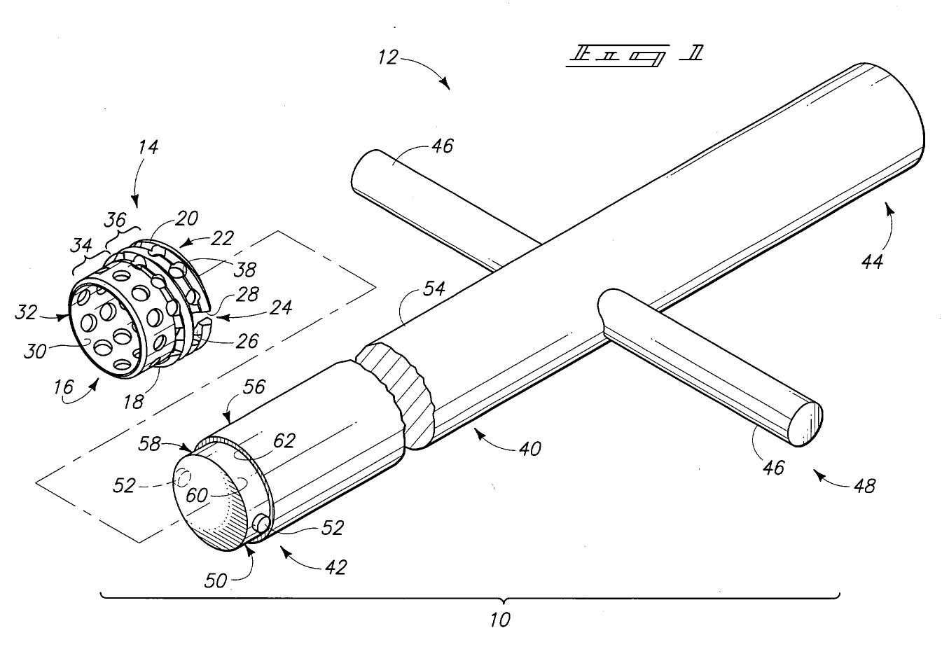

Reference will now be made to preferred embodiments of Applicant's invention. Two exemplary implementations are described below and depicted with reference to the drawings comprising various apparatus for inserting and removing bone joining devices such as spinal fusion devices between adjacent bone bodies. A first embodiment is shown and described below in a first configuration with reference generally to FIGS. 1-9. A second embodiment is shown and described below with reference to FIG. 10. While the invention is described by way of two preferred embodiments, it is understood that the description is not intended to limit the invention to these embodiments, but is intended to cover alternatives, equivalents, and modifications which may be broader than these embodiments such as are defined within the scope of the appended claims.

In an effort to prevent obscuring the invention at hand, only details germane to implementing the invention will be described in great detail, with presently understood peripheral details being incorporated by reference, as needed, as being presently understood in the art.

A preferred embodiment surgical implant system in accordance with the invention is first described with reference to FIGS. 1-9 and is identified by reference numeral 10. Such an implant system 10 is further described below with respect to an implant insertion instrument 12 and an accompanying spinal fusion device 14 that is configured to mate in inter-fitting engagement with instrument 12. Additionally, an optional configuration is shown with reference to surgical implant system 110, as identified in FIG. 10.

As shown in FIG. 1, surgical implant system 10 comprises implant insertion instrument 12 and spinal fusion device 14. Spinal fusion device 14 comprises a spinal fusion cage similar to that disclosed in U.S. Pat. No. 6,447,545 B1 to Bagby, issued Sep. 10, 2002, and incorporated herein by reference. However, spinal fusion device 14 includes the further additional features of special drive apertures 24 that inter-engage with drive pins 52 on instrument 12. The specific details of how drive apertures 24 interact with pins 52 will be described below in greater detail.

As shown in FIG. 1, spinal fusion device (or implant) 14 is shown having a cylindrical fusion body 16 with an outer portion (or surface) 18 that includes threads 24. Implant insertion instrument 12 is configured to mate in engagement with implant 14 to drive threads 20 of implant 14 between and into a pair of bone bodies (such as vertebral bodies), as identified and described below in greater detail with reference to FIGS. 5-9; and as further described in greater detail in U.S. Pat. No. 6,447,545 B1, previously incorporated by reference herein. However, implant 14 includes the specific improved features of drive aperture 24 and drive pins 52 which enable improved benefits when using instrument 12 to drive implant 14 between a pair of vertebral bodies (or other bone bodies).

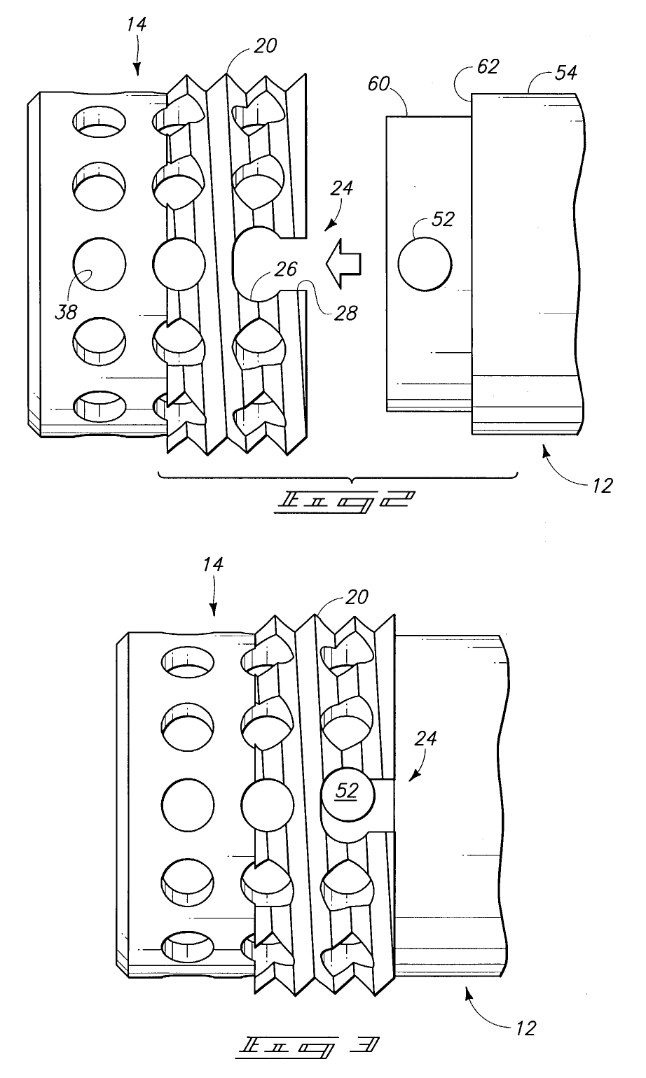

Implant 14 includes a trailing end portion (or trailing end) 22 in which a pair of drive apertures 24 are formed. Each drive aperture 24 includes an enlarged drive slot 26 that is spaced from trailing end 22 and communicates with trailing end 22 by way of a narrowed entrance slot 28 (See FIG. 2.). As shown in FIG. 2, enlarged drive slot 26 comprises an elongated slot having semicircular ends joined by an intermediate straight slot wherein the semicircular ends have radial outer surfaces that are separated by more than a diameter of a circle having the same radius as the combined semicircles. Secondly, narrowed entrance slot 28 is sized to provide a nominal clearance for pin 52 so pin 52 can enter slot 28 and be received within drive slot 26. However, slot 26 can have other geometric configurations, as long as slot 26 is wider than the width of entrance slot 28.

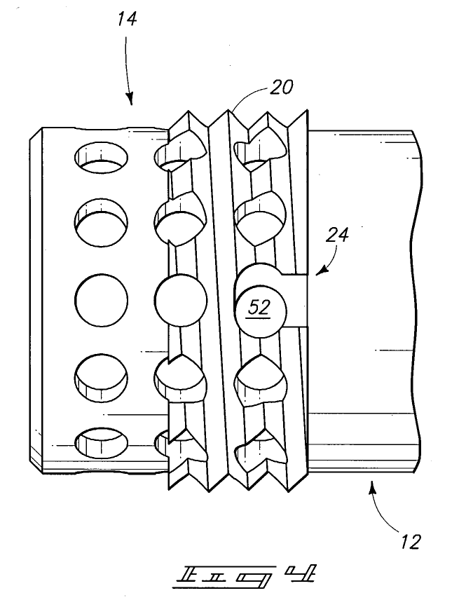

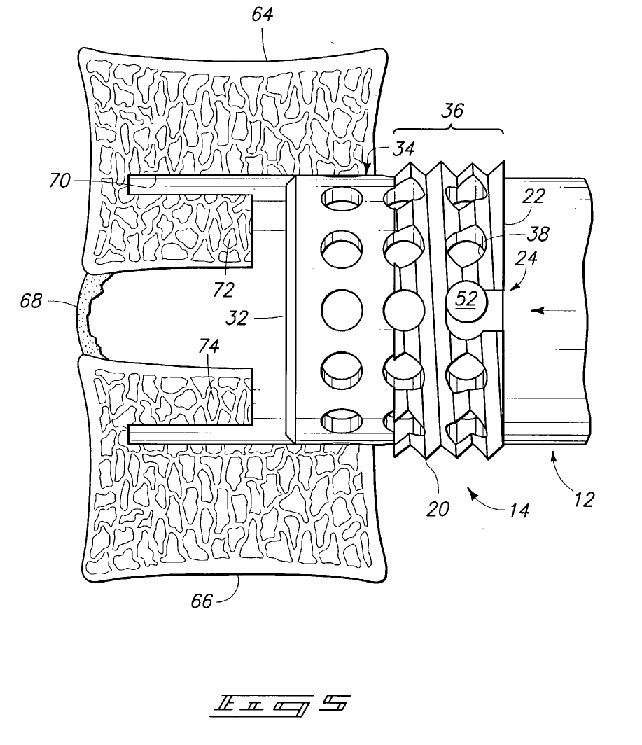

By studying FIGS. 2-4, the purpose for providing an enlarged drive slot 26 can clearly be seen. When loading implant 14 onto instrument 12, each pin 52 is axially received through a respective entrance slot 28 and into a corresponding drive slot 26. By slightly rotating implant 14 into the position shown in FIG. 3, pins 52 are received into an advancing end of each enlarged drive slot 26, which comprises a drive position that facilitates threading and driving (or torquing) of a right-handed thread 20 on implant 14 between a pair of bone bodies, as shown in FIG. 3. A second position is shown in FIG. 4, wherein implant 14 is rotated in an opposite direction so as to position each pin in a trailing end of each enlarged drive slot 26 which corresponds with a drive position that facilitates unthreading or removal (via torquing) of implant 14 from between a pair of adjacent bone bodies using instrument 12.

One benefit provided by the engaged positions depicted in FIGS. 3 and 4 is that, during torquing of instrument 12 while in the positions depicted in FIGS. 2 and 3, implant 14 does not tend to dislodge from instrument 12 because the position of pin 52 relative to the enlarged drive slot of drive aperture 24 mechanically retains (or fastens) implant 14 onto instrument 12. For example, during insertion of implant 14 between two vertebral bodies as shown in FIG. 3, pins 52 interlock in a forward drive position within drive aperture 24 which further stabilizes and locks implant 14 onto instrument 12 as instrument 12 is torqued to threadingly engage threads 20 between a pair of bone bodies. Once implant 14 has been positioned, instrument 12 is merely rotated in a reverse direction a few degrees so as to align pins 52 within the narrowed entrance slots 28 of drive aperture 24 to remove instrument 12 from implant 14.

In the event that it is desirable to remove implant 14, instrument 12 is similarly loaded as shown in FIG. 2, but instrument 12 is then rotated in a rearward drive position as depicted in FIG. 4 wherein pins 52 interlock within the enlarged drive slots of the drive apertures 24 (at an opposite end) which enables torquing of instrument 12 so as to unthread and remove implant 14 from between a pair of adjacent bone bodies. The engagement of pins 52 within the trailing ends of the enlarged drive slot of drive aperture 24 enables a user to apply axial distraction pressure on the implant 14 as instrument 12 is being torqued and pulled out to remove implant 14 because of the interlocking position of pins 52. When torquing implant 14 in a direction that unscrews the implant 14 from between a pair of vertebrae (untorquing) in combination with applying axial withdrawal pressure (by pulling back on instrument 12), the pins 52 remain in a locked position within the enlarged drive slots 26 due to frictional forces acting on the implant that resist rotation of the implant between the vertebrae. Such interlock of pins 52 within drive slots 26 enables application of axial withdrawal pressure on the implant 14 via instrument 12 (as depicted in FIG. 4).

Although the interlocking instrument and implant features of the present invention are illustrated according to one embodiment in FIG. 1, it is understood that such features can be used with a number of different implant and instrument constructions wherein threads are provided on a bone implant that requires torquing and untorquing in order to insert and remove, respectively, the bone implant from between a pair of bone bodies. Accordingly, it is understood that the features of the present invention can be utilized to insert and remove bone implants having different constructions than those shown in FIGS. 1-10, including any of a number of generally tubular bone cages and bone baskets having threaded portions on the outer surface, as is presently understood in the art.

As shown in FIG. 1, bore 30 of implant 14 provides an open leading end 32 in implant 14 about which a smooth insertion portion 34 is provided thereabout. Threads 20 provide an interlocking trailing portion 36 rearward of smooth insertion portion 34. As is understood in the art, implant 14 also includes a plurality of fenestrations 38 that contribute to enhance the realization of staged bony fusion and arthrodesis therethrough.

Implant insertion instrument 12 of FIG. 1 includes a drive body 40 having a leading end 42 and a trailing end 44. A pair of opposed handles 46 extend radially outwardly from a central portion of drive body 40 so as to provide a T-shaped handle portion 48. Handles 46 enable a user to torque the instrument 12 and implant 14, while also optionally providing axial insertion and removal forces (as desired). For example, axial insertion forces are applied via handles 46 to implant 14 when driving smooth insertion portion 34 into a cylindrical kerf 79 (see FIG. 5) during insertion of implant 14. However, during removal, axial removal forces are applied in combination with untorquing (reverse torquing) forces to remove implant 14. Such axial removal force is especially desired where bone threads between bone bodies have been damaged during insertion. In such a case, reverse torquing (unthreading) of implant 14 will often not drive the implant 14 out from between adjacent vertebrae without the additional application of an axial withdrawal force.

An implant-engaging surface 50 is provided along leading end 42 of drive body 40 for mating in coaxial engagement within bore 30 of implant 14 during insertion and removal of such implant from between a pair of bone bodies.

Implant engaging surface 50 is recessed radially inwardly from a cylindrical body 54 of drive body 40, along leading end 42. Such implant engaging surface 50 provides a recess within a cylindrical head portion 56 of cylindrical body 54 so as to provide a recessed diameter portion 58 thereabout. The recessed diameter portion 58 defines a recessed, cylindrical mounting surface 56 that mates in complementary, coaxial engagement within bore 30, upon assembly therein. Diameter portion 58 is recessed from head portion 56 so as to define a recessed, cylindrical mounting surface 60 that fits within bore 30 of implant 14 and also mates in abutment with a receiving shelf 62. When instrument 12 is assembled together with implant 14, receiving shelf 62 abuts in physical, circumferential engagement with trailing end 22 of implant 14 so as to provide a stable support and drive surface for implant 14. Likewise, a pair of pins 52 mate in interdigitating engagement within a pair of corresponding drive apertures 24 within implant 14.

Accordingly, drive body 40 includes leading end 42 with implant engaging surface 50 and a pair of axial drive pins 52 that radiate outwardly from leading end 42 of drive body 40. Such pins 52 are configured to engage with corresponding apertures 24 along a trailing end of implant 14. According to such one construction, implant engaging surface 50 comprises a cylindrical stud (or post) that is provided along leading end 42 of body 40. The stud provides a drive element that is integrally formed from an enlarged cylindrical portion of body 40; namely, cylindrical body 54. Receiving shelf 62 comprises a right angle cylindrical shelf that extends radially outwardly of such stud to the outer surface of cylindrical body 54.

As illustrated in FIGS. 2-4, implant 14 is of a construction that is initially axially inserted within a pair of bone bodies (as shown in FIG. 5), after which instrument 12 is rotated to position pins 52 into the configuration depicted in FIGS. 3 and 5. At such point, instrument 12 is torqued so as to drive threads 20 into the bone beds of vertebrae 64 and 66 as implant 14 is advanced into a cylindrical kerf 70. Kerf 70 is constructed by utilizing a series of machining operations using one or more hole saws within vertebrae 64 and 66 as discussed previously with reference to U.S. Pat. No. 6,447,545 B1, previously incorporated by reference.

As illustrated in FIG. 5, implant 14 is of a specific construction that utilizes bone projections (or peninsulas of living bone) 72 and 74 which extend within an inner bore of implant 14, along an open leading end 32. Preferably, a substantial portion of intervertebral disk 68 is removed or resected prior to insertion of implant 14 so as to further facilitate arthrodesis between vertebral bodies 64 and 66. Further details of the advantages of entrapping bone projections within an implant are described in U.S. Pat. No. 6,371,986 B1 to Bagby, issued on Apr. 16, 2000, and herein incorporated by reference.

As depicted in FIG. 5, intervertebral disk 68 is shown in somewhat simplified form. Pursuant to a typical insertion of an implant 14 that has a smooth insertion portion 34, distraction is typically imparted between vertebrae 64 and 66 so as to stretch intervertebral disk 68 (typically more than is illustrated in FIG. 5). However, it is not necessary that such distraction be imparted between vertebrae 64 and 66 during insertion of implant 14.

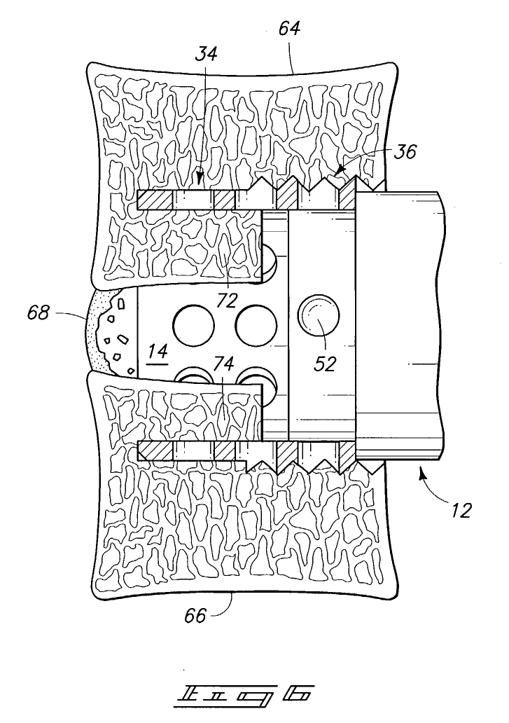

FIG. 6 illustrates implant 14 after being completely inserted between vertebral bodies 64 and 66 by torquing instrument 12 in a clockwise direction (from the perspective of a surgeon), as viewed by a user of instrument 12. This procedure assumes that implant 14 has right-handed threads.

Accordingly, FIG. 6 illustrates implant 14 shown completely driven into position between vertebral bodies 64 and 66 so as to entrap bone projections 72 and 74, and instantly fix adjacent vertebrae 64 and 66 together. Accordingly, vascularized, living bone is provided inside of implant 14 which has been found to accelerate bone-to-bone fusion and arthrodesis between vertebral bodies 64 and 66. However, it is understood that the improved insertion and removal features provided by the present invention can also be provided on more traditional spinal fusion cages having outer threads (or inner threads). It is not necessary that such bone projections be utilized to gain the advantages of the present invention.

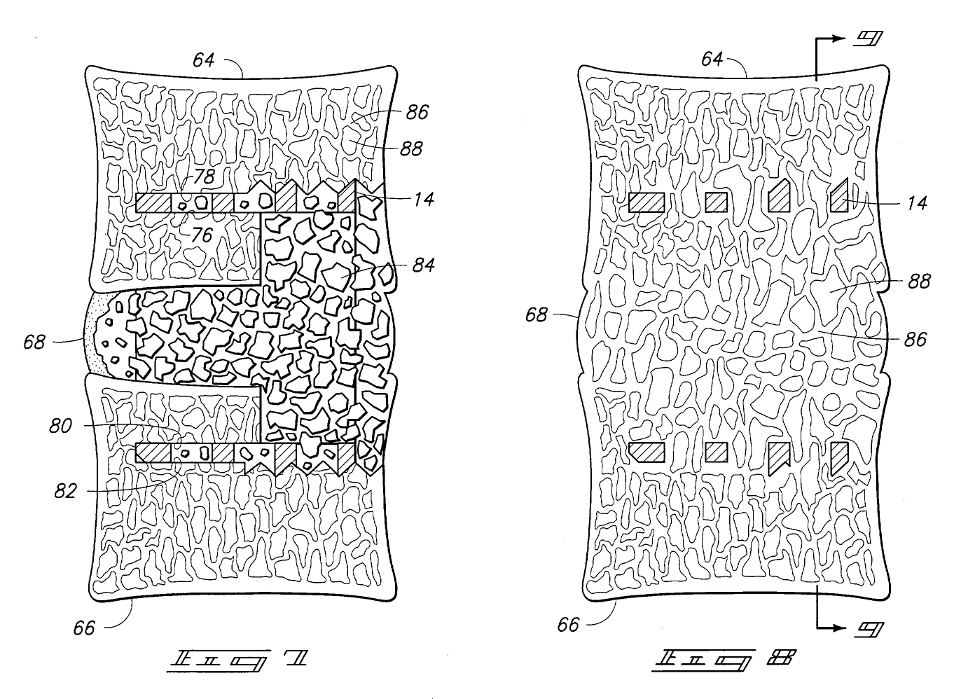

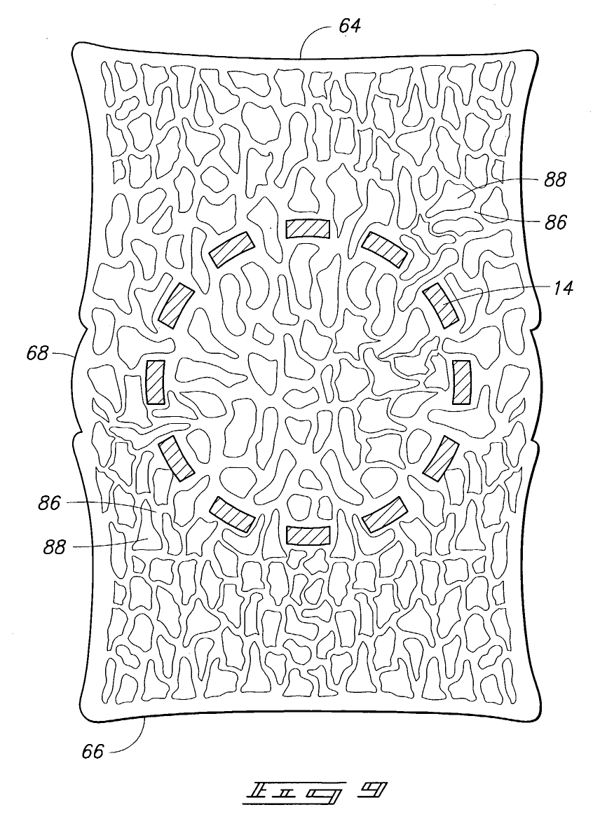

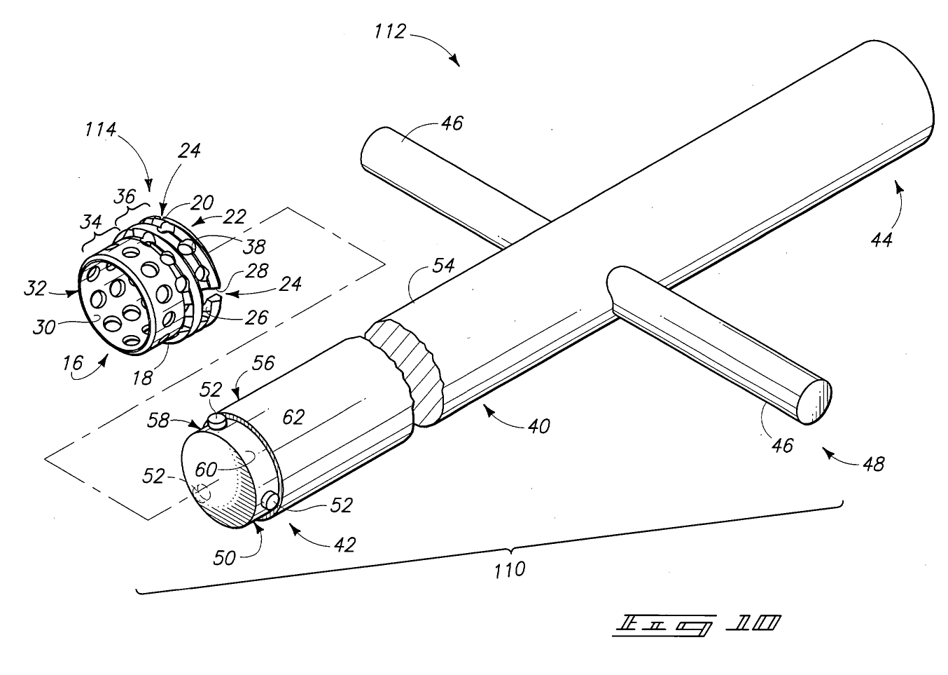

As shown in FIG. 7, bone chips 84, recovered when preparing the cylindrical kerf within vertebrae 64 and 66, facilitate early bone ingrowth and through-growth and eliminate the need to recover bone graft from a second surgical site. As shown in FIGS. 8-9 (as well as FIGS. 5-6), bone 86 and voids 88 are shown in an exaggerated, enlarged configuration for purposes of simplifying drawings. It is understood that the formation of trabeculae occurs on a finer scale than illustrated in such figures, and that the bone orients to optimize structural support of stresses and loads carried in the fused and arthrodized configuration of the unitary bone body provided by arthrodized vertebrae 64 and 66.

FIG. 8 illustrates stage stabilization and fusion via Wolff's Law, wherein bone remodeling and reorganization has further fixed and fused such adjacent vertebrae 64 and 66. The trabeculae relocate through fenestrations in order to form a mature strengthening of the trabeculae. Additional reorganization is provided by preparing bone beds that recess implant 14 within vertebrae 64 and 66, and by providing bone graft material thereabout at the time of implantation. Accordingly, additional bone reorganization is facilitated outside of implant 14.

More particularly, FIGS. 7 and 8 illustrate the reorganization of fused bone material through implant 14. Histological bone cell geometry is shown in greater detail, corresponding in time with complete bone remodeling, as shown in FIG. 8. Lacunae and canals, or voids, 88 are formed between the bone 86.

FIG. 9 is a coronal and diagrammatic view taken perpendicular to the view of FIG. 8 along line 9-9. In such view, bone cells have remodeled in order to form a definite elongated configuration extending between vertebrae 64 and 66. Such remodeled bone through-growth can be seen between fenestrations on some sides of a patient, occurring from cephalad to caudad as well as between fenestrations along a diagonal configuration of the patient, from cephalad to caudad.

FIG. 10 illustrates an alternative embodiment surgical implant system 110 having an implant insertion instrument 112 with three equally spaced-apart and radially extending pins 52 that mate with a set of three corresponding drive apertures 24 in spinal fusion (or cage) 114. Remaining features of system 110 are similar to those depicted in system 10 of FIGS. 1-9. It is understood that various other configurations can be utilized for pins 52 and drive apertures 24. For example, a single pin and aperture could be used. Alternatively, four, five, six or more pins and apertures could be utilized. Even furthermore, pins 52 do not necessarily need to take on a cylindrical configuration. For example, pins 52 could be constructed to be square in cross-sectional configuration, with aperture 24 having a correspondingly shaped enlarged drive slot portions having correspondingly configured enlarged drive slot portions that mate with the square cross-sectional configuration when the pins are rotated into the drive and removal positions within such slot.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

The invention claimed is:

1. Apparatus for driving an implant, comprising:
a drive body having a leading end with an implant engaging surface and a plurality of drive elements contiguous with the implant engaging surface, the drive elements radially outwardly extending from the implant engaging surface adjacent the leading end and configured to engage with corresponding apertures along a trailing end of an implant.

2. The apparatus of claim 1 wherein the drive body comprises a cylindrical body including a cylindrical proximal head portion including a recessed diameter portion along a leading end providing a recessed mounting surface and a receiving shelf.

3. The apparatus of claim 1 wherein the drive body further comprises a plurality of handles extending radially from a central portion of the body.

4. The apparatus of claim 4 wherein the plurality of handles provides a pair of handles that form a t-shaped handle portion of the body.

5. The apparatus of claim 1 wherein the implant engaging surface comprises a recessed, cylindrical mounting surface extending from the leading end of the body and a receiving shelf provided at a distal end of the mounting surface.

6. The apparatus of claim 1 wherein the implant engaging surface comprises a cylindrical stud at the leading end of the body communicating with an enlarged cylindrical portion of the body via a cylindrical shelf extending radially outwardly of the stud.

7. The apparatus of claim 1 wherein each stationary drive element comprises a radially extending drive arm.

8. The apparatus of claim 7 wherein the radially extending drive arm comprises a cylindrical pin.

9. The apparatus of claim 1 wherein the drive body includes an axial cylinder with the leading end, a central shaft, and a trailing end, the leading end forming a cylindrical proximal head portion including a recessed diameter portion along a leading end, a recessed mounting surface, a receiving shelf, and a pair of drive pins circumferentially opposite one another and extending radially outwardly of the recessed diameter portion, and the central shaft including a pair of radially outwardly extending drive arms opposite one another.

10. The apparatus of claim 1 wherein the implant engaging surface comprises a cylindrical post configured to be received coaxially within an inner cylindrical surface provided at a trailing end of an implant.

11. A spinal fusion device, comprising:
a cylindrical fusion body having an outer surface including threads, a trailing portion with an open trailing end, and a plurality of drive apertures formed in an inner surface of the open trailing end, each drive aperture having an enlarged drive slot spaced from the trailing end and a narrowed entrance slot provided in the trailing end and communicating with the enlarged drive slot.

12. The spinal fusion device of claim 11 wherein the enlarged drive slot comprises an oblique slot extending along a circumferential portion of the body.

13. The spinal fusion device of claim 11 further comprising an internal bore communicating with the open trailing end.

14. The spinal fusion device of claim 13 wherein the internal bore comprises a cylindrical bore.

15. The spinal fusion device of claim 11 wherein each drive aperture extends from the outer surface of the fusion body to the inner surface of the open trailing end.

16. The spinal fusion device of claim 11 wherein a pair of opposed drive apertures are provided each on opposite sides of the trailing edge.

17. The spinal fusion device of claim 11 wherein the fusion body includes a smooth insertion portion adjacent a leading end and the threads are provided between the smooth insertion portion and the trailing end.
